(19) United States
(12) Patent Application Publication
(10) Pub. No.: US 2008/0294072 A1
(43) Pub. Date: Nov. 27, 2008

(54) MESOTHERAPY WITH ULTRASOUND

(75) Inventor: Charles E. Crutchfield, III, Eagan, MN (US)

Correspondence Address: BRIGGS AND MORGAN P.A., 2200 IDS CENTER, 80 SOUTH 8TH ST, MINNEAPOLIS, MN, 55402, US

(73) Assignee: Crutchfield Dermatology, Eagan, MN (US)

(21) Appl. No.: 12/125,616

(22) Filed: May 22, 2008

Related U.S. Application Data
(60) Provisional application No. 60/931,605, filed on May 24, 2007.

(51) Int. Cl. A61N 7/00; A61M 5/00

(52) U.S. Cl. 601/2; 604/22

(57) ABSTRACT

Mesotherapy with ultrasound delivered at 1 to 3 Mhz for five minutes before and five minutes after a phosphatidylcholine-deoxycholate solution is injected into the areas of unwanted fat.

### CROSS-REFERENCE TO RELATED APPLICATION

The present application is based on and claims the benefit of U.S. provisional patent application Ser. No. 60/931,605, filed Jun. 24, 2007, the content of which is hereby incorporated by reference in its entirety.

### BACKGROUND OF THE INVENTION

The present invention relates to an overall system for the dissolution of unwanted fat in the human body. Lipodissolve or mesotherapy is a technique in which phosphatidylcholine and deoxycholate are injected in a liquid form into areas of unwanted fat such as the tummy, double chins, love handles, and saddlebags on the thighs that causes an inflammatory response and dissolution of fat cells. This causes an overall reduction in fat. However, there is a need for improving the technique by use of ultrasound. LipoDissolve is a minimally invasive medical procedure that breaks down fat cells in specific areas of the body. Small amounts of lecithin, a soybean extract (commonly used as an emulsifying agent in chocolate bars), are injected into unwanted areas of fat such as the tummy, love handles, double chins and saddle bags. This will cause the fat cells to dissolve and the body's natural cleansing system metabolizes and eliminates the waste products out of the system.

The technique has become increasingly popular in the United States over recent years. LipoDissolve is a specific type of treatment known generally as “Mesotherapy” that was first developed in France in the 1950's.

### SUMMARY OF THE INVENTION

The present invention improves upon mesotherapy by delivering ultrasound at 1 to 3 Mhz for five minutes before and five minutes after the phosphatidylcholine-deoxycholate solution is injected into the areas of unwanted fat.

### BRIEF DESCRIPTION OF DRAWINGS

FIG. 1 is a photograph of an area of adipose tissue before treatment using the current method.

FIG. 2 is a photograph of the same area after treatment using the current method.

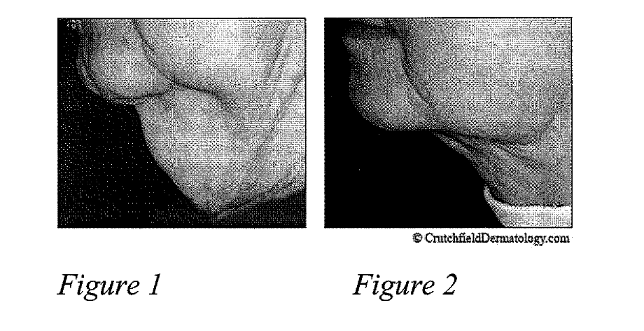

### DETAILED DESCRIPTION OF THE PREFERRED EMBODIMENT

The present invention is a new method for the dissolution of unwanted fat in the human body that employs ultrasound massage both before and after the LipoDissolve treatments. Some LipoDissolve practitioners use ultrasound before, some after, and some not at all. The present invention employs ultrasound massage both before and after LipoDissolve treatments. When ultrasound is used before, it makes the adipose tissue more receptive to the treatments, and when ultrasound is used after the treatments, it makes sure that the solution is distributed evenly, reduces post-treatment edema, and increases patient comfort. The method is particularly indicated for unwanted areas of fat, especially those that have not responded to diet and exercise.

The injections of many agents into the skin and subcuticular regions have been shown to be beneficial. Most recently, the injection of multiple substances has been shown to actually cause fat dissolution also known as injection lipolysis. In this patent, we are going to use a unique combination of ultrasound massage performed before the treatment and after the treatment, utilizing an ultrasound therapy device, as an integral overall program for the most effective and comfortable form of injection lipolysis. Ultrasound massage will be performed using an ultrasound machine with a megahertz range of 1-10 MHz with the ideal range being 1-3 MHz. The massage will be performed on the treatment area with the treatment range of 1 to 15 minutes with an ideal target range of 3-7 minutes. This treatment will be performed on the target treatment area both before and after the injection session. The machine used is a Mettler 740; however, other ultrasound machines, as listed, are available. The injections themselves will consist of a solution containing phosphatidylcholine (lecithin) at a concentration range of 5-100 mg/cc with an ideal concentration of 25 mg/cc. As a stabilizing factor), the injections will contain deoxycholate in a concentration range of 1-10% with an ideal concentration of 2%. The injections will include a solution volume target range of 3-50 cc/7 sq. cm surface area of skin with an ideal range of 10-30 cc/35 sq. cm. Injections will be performed directly into the subcutaneous adipose tissue. Once again, injection lipolysis with ultrasound pre and post treatment will be performed as outlined above.

The FDA has not officially approved this treatment but it is currently under investigation.

Experiments conducted using the method of the present invention for the reduction of unwanted fat show a success rate approaching 90%.

The present invention may be embodied in other specific forms without departing from the spirit or essential attributes thereof, and it is therefore desired that the present embodiment be considered in all respects as illustrative and not restrictive.

1. A method of mesotherapy with ultrasound for unwanted human fat cell removal comprising the following steps:
a.) providing ultrasound massage with an ultrasound machine with a megahertz range of 1-10 Mhz for 1 to 15 minutes on the unwanted human fat cells;
b.) subcutaneously injecting into the fat cells with a solution containing phosphaltidylcholine at a concentration in a range of 5 to 100 mg/cc; and
c.) providing ultrasound massage with an ultrasound machine with a megahertz range of 1-10 Mhz for 1 to 15 minutes on the injected human fat cells.

2. The method of claim 1 wherein the solution further comprises deoxycholate in a concentrated range of 1%-10%.

3. The method of claim 1 wherein the solution volume is in a target range of 3-50 cc/7 cm² surface area over the fat cells.

4. The method of claim 1 wherein the ultrasound megahertz range is ideally narrowed to 1-3 Mhz.

5. The method of claim 1 wherein the solution of phosphaltidylcholine is approximately 25 mg/cc.

6. The method of claim 1 wherein the ultrasound massage is ideally narrowed to 3-7 minutes.

7. The method of claim 1 wherein the solution further comprises deoxycholate in a concentration of about 2%.

8. The method of claim 1 wherein the solution volume is in a target range of 10-30 cc/35 cm² surface area over the fat cells.
